(19) United States
(12) Patent Application Publication
(10) Pub. No.: US 2022/0288371 A1
(43) Pub. Date: Sep. 15, 2022

(54) Microarray Applicator and Method for Displacing a Plunger Acting Upon the Microarray

(71) Applicant: LTS Lohmann Therapie-Systeme AG, Andemach (DE)

(72) Inventors: Sebastian Scherr, Neuhäusel (DE); Miriam Britten, Bonn (DE); Maurice Tak, Schüttorf (DE); Marianne Meijerink, Enschede (NL)

(21) Appl. No.: 17/638,119

(22) PCT Filed: Aug. 25, 2020
(86) PCT No.: PCT/EP2020/073670

§ 371 Date: Feb. 24, 2022

(30) Foreign Application Priority Data
Aug. 27, 2019 (DE) 10 2019 122 948.0
Apr. 6, 2020 (DE) 10 2020 109 563.5

(51) Int. Cl. A61M 37/00

(52) U.S. Cl. CPC A61M 37/0015; A61M 2037/0023; A61M 2037/0061

(57) ABSTRACT

An applicator for applying a microarray, including a housing, a plunger which is arranged in the housing in a linearly movable manner, a holding device for receiving a microarray device, a pressing element for moving the plunger in order to act on a microarray device received by the holding device, and an actuation device which holds the plunger in a first position and releases the plunger for movement. The plunger is released by the actuation device only when the at least two actuators are actuated. Further, an application system with such an applicator and a microarray device and to a method for displacing a plunger are included. The plunger is held in a first position against a pretension, and the plunger is released for movement by the pretension upon carrying out at least two separate actuations, where the at least two actuations must be carried out for the release process.

### CROSS-REFERENCE TO RELATED APPLICATIONS

This application is the U.S. national phase of International Application No. PCT/EP2020/073670 filed Aug. 25, 2020, and claims priority to German Patent Application Nos. 10 2019 122 948.0 filed Aug. 27, 2019, and 10 2020 109 563.5 filed Apr. 6, 2020, the disclosures of which are hereby incorporated by reference in their entirety.

### BACKGROUND OF THE DISCLOSURE

The disclosure relates to an applicator for applying a microarray. Further, the disclosure relates to an application system for applying a microarray. Furthermore, the disclosure relates to a method for displacing a plunger.

### DESCRIPTION OF RELATED ART

Microarrays comprise a plurality of microneedles which are usually arranged on a carrier element, such as a patch, a band-aid or the like, or are connected to a carrier element. Such microarrays comprise a large number of microneedles. The needles have a small length such that, when the microneedles are pressed into the skin of a patient, the needles only penetrate the skin to such an extent that nerves and blood vessels are not contacted by the needle points, if possible. The microneedles contain an active ingredient or a medicament. The corresponding active ingredient can be applied to a surface of the needle or arranged inside the needle. Preferably, the needles are made from a material dissolving in the skin of a patient.

When microarrays are applied to human skin, the problem arises that insertion of the microneedles into the skin must be reproducible for ensuring in particular reliable dispensing of a medicament. Also, insertion of the microneedles must be independent of the user or the patient since otherwise reproducibility would not be ensured. In particular, insertion of the microneedles should also be independent of the type of skin such that a specific penetration depth is always ensured.

Microarrays are generally stored in packages and/or carrier devices for protecting them against environmental influences, such as physical effects and/or contaminations, as well as for transport purposes.

A carrier concept for microarrays is illustrated in DE 10 2019 200 561 A, for example.

Another problem arising in conjunction with microarrays is that, when users apply microarrays in microarray carrier devices, they encounter initial problems, in particular they do not know how to apply such microarrays. This is due to the fact that interfaces via which various microarray carrier devices are acted upon and/or necessary types of action, in particular acting force, are not unambiguous.

### SUMMARY OF THE DISCLOSURE

It is an object of the disclosure to provide an applicator for applying a microarray by means of which handling of the microarray is improved. Another independent object of the disclosure is to provide an application system for applying a microarray by means of which handling by the user is improved. Furthermore, an additional independent object of the disclosure is to provide a method for displacing a plunger which allows for reliable plunger action.

According to the disclosure, the objects are achieved with an applicator having the features of claim 1, an application system having the features of claim 14 as well as a method for displacing a plunger having the features of claim 15.

The applicator according to the disclosure for applying a microarray comprises a housing in which a plunger is arranged in a linearly movable manner. Preferably, the applicator comprises a plastic material, in particular is made from the same. Preferably, the applicator comprises, in particular besides a plastic material, a metal, for example metallic reinforcing elements, such as bars. Particularly preferably, the applicator, in particular all elements of the applicator, are injection-molded components. Preferably, the housing has a cylindric shape. Particularly preferably, this is a hollow cylindrical shape. In particular, this is a circular cylinder or a rectangular, in particular square cylinder. Further, a holding device for receiving a microarray is in particular connected to the housing or arranged at the same.

Preferably, the microarray device is a microarray carrier device. Particularly preferably, the microarray device is a carrier as defined in DE 10 2019 200 561 A, preferably in one of the embodiments and/or the claims, in particular claim 1. In particular, the microarray device is a microarray carrier disk.

Furthermore, the applicator comprises a pressing element for displacing the plunger. In particular, the pressing element is an element which is subjected to pressure or tension. Particularly preferably, this is a pretensioning device, such as a spring, particularly preferably a coil spring, for example. Preferably, the plunger is displaced by means of the pressing element for acting upon a microarray device received by the holding device. Furthermore, the applicator comprises an actuating device which holds the plunger in a first position and, by actuation by means of the actuating device, releases the plunger for displacement by the pressing element. The first position is in particular an initial position, wherein it is preferred that this is an internal position inside the housing at which the plunger is located. When the plunger has been displaced, in particular into the maximum deflected position, the plunger is preferably in a second position which is also referred to as a deflecting position. Preferably, the plunger has a conical or pyramidal or part-spherical shape such that the plunger can in particular act upon a negative-opposite surface of a microarray receiving portion, preferably a microarray carrier plunger, for allowing for an optimum acting force. Preferably, the shape of the plunger is selected such that said plunger can penetrate or bend a film, in particular a sealing film, of a microarray device. The actuating device comprises at least two separate actuators. The actuators are in particular mechanical actuators, i.e. elements which are actuated by mechanical action. Alternatively or additionally to the configuration as a mechanical actuator, a configuration as an electric actuator is also feasible. The configuration of the applicator, in particular the configuration of the actuating device, is such that the plunger is released by the actuating device only upon actuation of the at least two actuators. Here, it is particularly preferred that the release is effected only if a cumulative actuation of the at least two actuators takes place. Here, cumulative means simultaneous. Simultaneous/cumulative in particular means common actuation, wherein this in particular means a simultaneous and/or consecutive actuation. Simultaneous in particular means that actuation essentially takes place at the same moment. For example, consecutive means that first one of the actuators is actuated, for example a lever or a slider is moved into an actuating position and thus unlocking is effected, for example. Then the other actuator is actuated, in particular an actuator knob is pressed, such that triggering is effected, for example. In other words, it is preferred that release is effected only when unlocking and triggering have been performed.

Here, the configuration including the pressing element allows in a particularly advantageous manner for a definition of the application force to be carried out in advance, thus making a reproducible application possible. For this purpose, it is preferred that the pressing element is selected and/or adjusted and/or configured such that an optimum application force is triggered. By means of two separate actuators it is in particular possible in an advantageous manner to realize a kind of protective function as well as a kind of triggering function. In other words, a kind of unlocking in the style of a gun can be realized such that a triggering function is feasible only after unlocking.

The applicator according to the disclosure in particular offers the advantage that reproducibility of insertion of the microarray is improved. Another advantage is that due to the specified actuation of the at least two actuators, in particular unlocking and triggering, the transport safety is improved and/or unintentional activation is prevented. Furthermore, it is particularly advantageous that an optimum, in particular minimum pressing force is preferably ensured in the direction of the application site in order to ensure bonding and a sufficient holding force during the triggering pulse duration.

Preferably, the pressing element is configured such that it is not possible that, after the plunger has been displaced by the pressing element, in particular re-tensioning can be manually performed by a user but preferably only by means of an implement, such as a tool, for example a tensioning tool. Here, it is preferred that the pressing element has such a large spring force and/or spring constant that manual tensioning is not possible. Preferably, the applicator is advantageously configured such that in particular a manual reuse is prevented in particular by the spring force and/or spring constant of the pressing element.

According to a preferred embodiment, the actuation device comprises a force triggering element. The force triggering element is in particular a first actuator of the actuating device. The force triggering element is configured such that, when a defined force is applied in particular in the direction of the application site, actuation takes place. Here, application of a defined force means that either exactly the predefined force and/or a larger force than the predefined forced must be applied for triggering an actuation. In other words, the force triggering element preferably is a triggering element having a force threshold, wherein, when a force exceeding the force threshold is applied, actuation takes place. It is particularly preferred that the force triggering element comprises a rated-break-point device and/or a flexible resilient element and/or a pretensioning device for realizing the force definition. Also, it is possible that the actuating device comprises a plurality of force triggering elements. For example, it is possible that the first and the second actuator each are a force triggering element. The defined force, in particular the force threshold, preferably amounts to approximately 2 kg, preferably 2.5 kg, particularly preferably 3 kg. Preferably, the defined force, in particular the force threshold, amounts to approximately 20 N, preferably approximately 25 N, particularly preferably approximately 30 N.

Preferably, the actuating device comprises at least one operating element which is actuated when being acted upon. It is particularly preferred that a second actuator is the operating element. On other hand, it is also possible that the first and the second actuator are operating elements, for example. The at least one operating element is in particular a preferably technical command controller which can be manipulated by a user for triggering an actuation.

The force triggering element and/or the operating element in particular comprise at least one of the following devices, wherein it is preferred that the force triggering element and/or the operating element are constituted by one of the following devices: pressing and/or rotary knob or lever or slider or switch. Here, pressing knob means a haptically movable element which is preferably adapted to be pressed in and/or pulled out.

According to a preferred embodiment, the force triggering element and the operating element are connected to each other. It is particularly preferred that this is a one-piece, also referred to as integral, connection. For example, it is preferably possible that the force triggering element and the operating element are jointly configured as a rotary/pressing knob. Such a rotary/pressing knob can thus preferably be pressed in and hence assume the function of the force triggering element on the one hand, and be rotated and hence assume the function of the operating element on the other hand. Preferably, other or similar combinations are feasible, such as a pressing/sliding knob, for example, wherein such a knob must first be slid into a position and can be pressed in this position, for example.

According to a preferred embodiment, the actuating device comprises a coupling mechanism. It is particularly preferred that this is a form-locking and/or force-locking coupling mechanism. The coupling mechanism is configured such that it couples the plunger, in a first state, with the housing and decouples it, in a second state, from the housing for allowing the plunger to be displaced. Hence, it is preferred that the plunger, which is first preferably coupled inside the housing, is held by the coupling mechanism. When the coupling mechanism is actuated or activated, in particular when the decoupling function of the coupling mechanism is triggered, decoupling or release is effected such that it is possible to displace the plunger.

Preferably, the actuating device comprises a locking mechanism. The locking mechanism is configured such that it blocks the coupling mechanism in a first, preferably deactivated state and unblocks it in a second, preferably activated state such that the coupling mechanism can allow the plunger to be displaced. In other words, it is thus preferred that the locking mechanism moves the applicator, particularly preferably the coupling mechanism, from a stable into an unstable state. Consequently, the locking mechanism is in particular configured such that it first prevents activation of the coupling mechanism, i.e. decoupling, and releases it only upon activation of the locking mechanism, i.e. upon unlocking.

In a force-locking configuration of the coupling mechanism it is preferred that the coupling mechanism preferably pressingly abuts on the plunger and thus prevents, via friction, movement of the plunger in the first state. Here, it is preferred that contact with the coupling mechanism or pressing the coupling mechanism to the plunger is released upon decoupling. In a force-locking configuration of the coupling mechanism it is preferred that the coupling mechanism comprises a hook device and/or a retaining pawl device and/or a template device and is in particular constituted by the same. Here, it is particularly preferred that these devices couple the plunger, in the first state, to the housing or block it. The hook device and/or retaining pawl device can hence selectively couple the plunger to the housing, for example. In a configuration as a template device it is preferred that the coupling mechanism comprises a template through which a corresponding counterpart of the plunger, in the first state, cannot be fitted and then, upon uncoupling, for example displacing the template device, can be passed through.

In a form-locking and/or force-locking configuration of the locking mechanism it is preferred that the configuration is based on the form-locking and/or force-locking configuration of the coupling mechanism. In contrast to the configuration of the coupling mechanism it is, however, preferred that the locking mechanism does not act upon the plunger but upon the coupling mechanism, in particular blocks the same. If the locking mechanism comprises a hook or is constituted by the same, for example, it is preferred that the hook, in the locked state, blocks the coupling mechanism, i.e. is in particular hookingly connected to the same. In the unblocked state, it is preferred that the hook is unhooked from the coupling mechanism such that the coupling mechanism can be decoupled.

Preferably, the force triggering element actuates the decoupling mechanism or the locking mechanism.

In a preferred configuration, the operating element actuates the decoupling mechanism or the locking mechanism.

It is particularly preferred that, when the force triggering element actuates the decoupling mechanism, the operating element actuates the locking mechanism, or vice versa.

Preferably, the holding device is reversibly detachably connectable to the microarray device or that the holding device is irreversibly connectable to the microarray device. The reversibly detachable holding device allows the applicator to be reused. In the case of a reversibly detachable holding device it is preferred that the holding device comprises at least one flexible holding arm and/or catching device and/or at least one a snap-in hook and is particularly preferably constituted by the same. If the holding device is irreversibly connectable, it is in particular a disposable applicator, wherein it is particularly preferred that the holding device comprises a bayonet device, in particular is constituted by the same. Preferably, the holding device comprises a holding device operating member for reversibly detaching the microarray device.

According to a preferred embodiment, the applicator comprises a protecting device which, in a first position, shields at least a portion of the microarray device from the environment and, in a second position, releases it for application purposes. It is particularly preferred that the protecting device is movable relative to the holding device. Preferably, the protecting device shields at least one microarray of the microarray device and/or an adhesive layer of the microarray device provided for adhesive connection to the skin. In other words, the protecting device preferably is a kind of shield which preferably is foldable and/or retractable.

In particular, the protecting device comprises a protecting collar and is preferably constituted by the same. In a preferred configuration, the protecting collar, in the first position, is held by a pretensioning device. Thus, it is preferred that the pretensioning device prevents the protecting collar, in the first position, from moving into the second position. For example, the force application counter to the pretensioning direction allows the protecting collar to be moved, in particular retracted, from the first position into the second position, wherein at least a portion of the microarray device is released for application purposes.

Preferably, the applicator comprises a reversible or an irreversible blocking device. The blocking device is preferably configured such that it prevents the applicator from being reused. Here, it is particularly preferred that the blocking device fixes the plunger in the displaced position, in particular in the deflected position, and/or blocks the actuating device. For this purpose, it is preferred that the blocking device comprises a catching device and/or a snap-in hook or the like, for example.

The application system according to the disclosure is an application system for applying a microarray. The application system comprises an applicator as per one or more of the aforementioned definitions. Further, the application system comprises a microarray device having at least one microarray. The microarray device is preferably configured as a microarray carrier disk, in particular as described in DE 10 2019 200 561 A, particularly preferably as per the embodiments and/or the claims, in particular claim 1 described there. The holding device in particular catchingly receives the microarray device. Particularly preferably, receiving is effected via a reversible or irreversible connection of the holding device to the microarray device. With regard to the connection, it is preferred that the holding device comprises at least one flexible holding arm and/or catching device and/or snap-in hook device and/or bayonet device and is in particular constituted by the same.

The method according to the disclosure is a method for displacing a plunger, in particular an applicator plunger. The method includes the method step of holding the plunger in a first position against a pretension. In at least two separate, in particular mechanical actuation steps the plunger is released such that the plunger is displaced due to the pretension. The at least two actuating steps for releasing purposes must preferably be performed cumulatively and thus in particular simultaneously or consecutively. In other words, preferably according to alternative definitions, the plunger is released only after a step of unlocking and a subsequent step of triggering.

It is particularly preferred that the method is performed by an applicator having one or more features of the applicator defined above or an application system. Further, it is preferably possible that the method is supplemented by one or more of the features, preferably method features, defined above. Preferably, the application system is placed upon the application site before or after the step of unlocking or actuating the first actuator.

### BRIEF DESCRIPTION OF THE DRAWINGS

Hereunder the disclosure will be explained in detail on the basis of preferred embodiments with reference to the accompanying drawings in which:

FIG. 1 shows a schematic perspective view of an embodiment of a carrier,

FIG. 2a shows a schematic sectional view of the carrier of FIG. 1 along II in an initial position,

FIG. 2b shows the carrier of FIG. 2a in an application position,

FIG. 3 shows a schematic sectional view of another embodiment of a carrier,

FIG. 4 shows a schematic sectional view of another embodiment of a carrier,

FIG. 5a shows a schematic sectional view of another embodiment of a carrier,

FIG. 5b shows the carrier of FIG. 5b in an application position,

FIG. 6 shows a schematic sectional view of another embodiment of a carrier,

FIG. 7 shows a perspective view of an embodiment of an application system according to the disclosure with an embodiment of an applicator according to the disclosure,

FIGS. 8a show perspective sectional views of the application system of FIGS. 7 and 8b in two states,

FIG. 9 shows schematic sectional views of another embodiment of an application system according to the disclosure with another embodiment of an applicator according to the disclosure in the style of the respective embodiment of FIG. 7 in various states,

FIG. 10 shows a perspective view of another embodiment of an application system according to the disclosure with another embodiment of an applicator according to the disclosure,

FIGS. 11a show perspective sectional views of the application system of FIGS. 10 and 11b in two states,

FIG. 12 shows schematic sectional views of another embodiment of an application system according to the disclosure with another embodiment of an applicator according to the disclosure in the style of the respective embodiment of FIG. 10 in various states,

FIG. 13 shows a perspective view of another embodiment of an application system according to the disclosure with another embodiment of an applicator according to the disclosure,

FIGS. 14a show perspective sectional views of the application system of FIGS. 13 and 14b in three states,

FIG. 15 shows schematic sectional views of another embodiment of an application system according to the disclosure with another embodiment of an applicator according to the disclosure in the style of the respective embodiment of FIG. 13 in various states,

FIG. 16 shows a perspective view of another embodiment of an application system according to the disclosure with another embodiment of an applicator according to the disclosure,

FIGS. 17a show perspective sectional views of the application system of FIGS. 16 and 17b in two states,

FIG. 18 shows schematic sectional views of another embodiment of an application system according to the disclosure with another embodiment of an applicator according to the disclosure in the style of the respective embodiment of FIG. 16 in various states, and

FIG. 19 shows schematic sectional views of another embodiment of an application system according to the disclosure with another embodiment of an applicator according to the disclosure.

### DETAILED DESCRIPTION

In the Figures similar or identical components or elements are identified by the same reference numerals. In particular, for reasons of clarity, elements already identified are preferably not provided with reference numeral in all Figures.

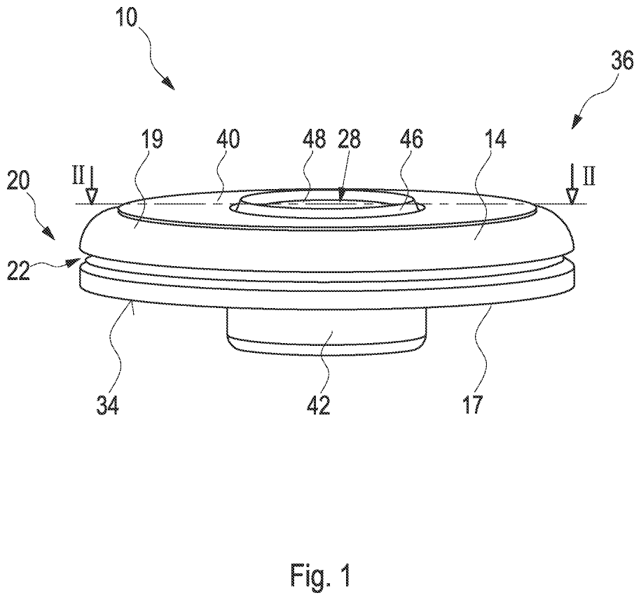

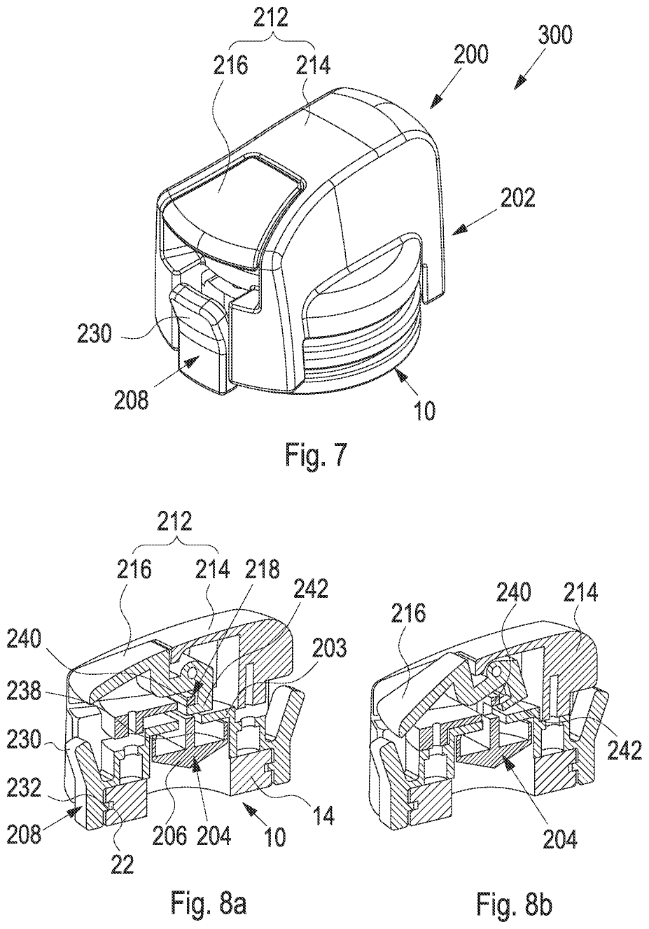

FIG. 1 show a microarray device configured as microarray carrier device 10, hereinafter referred to as carrier 10. The carrier 10 of FIG. 1 comprises a carrier housing 14. On the outside the carrier housing 14 comprises a circumferential groove 22 serving as a connecting means for an applicator 200 (see FIG. 7, for example).

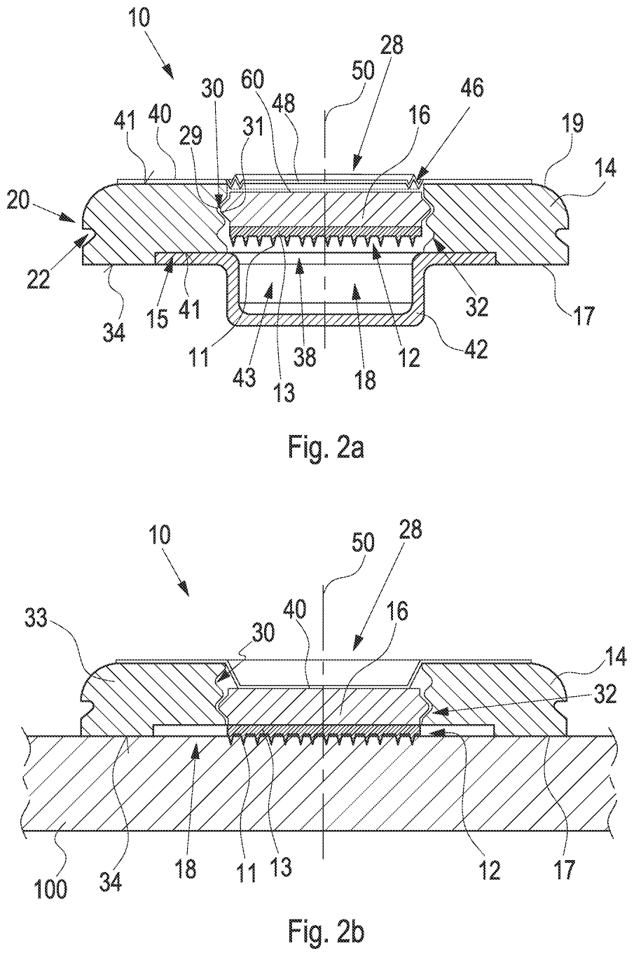

At the proximal end 19 the carrier housing 14 comprises an access opening 28 (see FIG. 2a) through which the plunger 204, also referred to as a piston, of an applicator 200 (see FIG. 7, for example) accesses the inside 38 of the carrier housing (see FIG. 2a) or acts upon the same, for example.

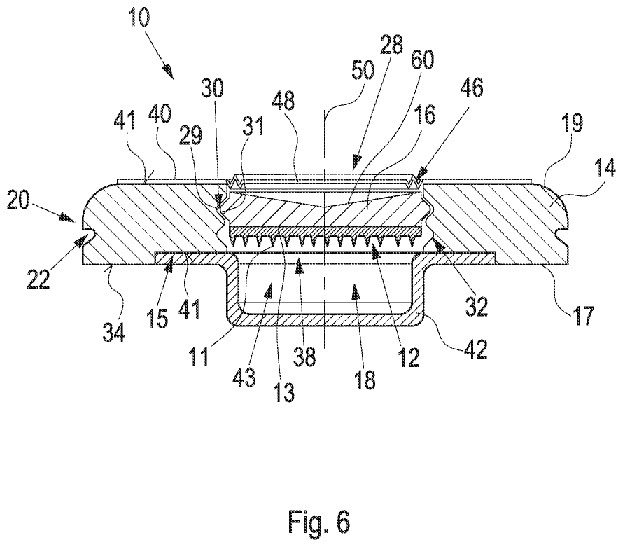

The access opening 28 is closed towards the environment by a film 40 forming part of a sterile barrier 36. Preferably, the film 40 is connected to the carrier housing 14 by means of an adhesive and/or weld connection 41. The film 40 comprises a flexible region 46 such that the plunger 204 of an applicator 200 (see FIG. 7, for example) can distally press-in the inside of the film 48 when the plunger 204 accesses the inside 38 of the carrier housing, for example. Preferably, an applicator 200 can thus move the carrier plunger 16, however, in the non-triggered state, there is still a sterile insulation towards the environment with respect to the illustrated upper side. Instead of the flexible configuration, it is also possible that the film 40, in particular in the region 46, is of a fragile configuration such that, when the carrier plunger 16 moves, an applicator 200 can provoke tearing of the film 40, for example. The action of a plunger 204 of an applicator (see FIG. 7, for example), which in particular results in a movement and/or displacement of the carrier plunger 16, preferably takes place on the carrier plunger acting area 60 at the proximal end of the carrier plunger 16. The carrier plunger acting area 60 is illustrated as a plane configuration. However, other configurations of the carrier plunger acting area 60 are also feasible, for example a negatively conical configuration (see FIG. 6) or a positively conical configuration or a negatively or positively pyramidal configuration or a configuration having a smooth, sharp or serrated edge or having a tip.

At the distal end 17 a blister 42 is connected to the carrier housing 14. As illustrated in FIG. 2a, for this purpose a recess 15 can be provided in the carrier housing 14 which receives the blister 42. Additionally or alternatively, the connection between the blister 42 and the carrier housing 14 can be provided at the connecting point 41, in particular configured as a releasable adhesive connection.

FIG. 2a shows the carrier plunger 16 in an initial position. Here, the carrier plunger 16 is fixed in a proximal position relative to the carrier housing by means of a releasing device 30. This fixing is realized by engagement of the bead 31 of the carrier plunger 16 with the groove 29 of the carrier housing 14. Here, the bead 31 and/or the groove 29 are preferably flexible, wherein the carrier housing 14 and/or the carrier plunger 16 are in particular at least essentially dimensionally stable.

At the distal end the carrier plunger 16 has connected thereto a microarray 12. For this purpose, the patch 13 of the microarray 12 is in particular adhesively connected to the carrier plunger. The patch 13 of the microarray 12 comprises distally extending microneedles 11. In the initial position illustrated in FIG. 2a, the microarray is located inside the carrier housing 14. The blister 42 and the film 40 create a sterile barrier 36 for insulating the microarray 12 towards the environment.

The inside 38 of the carrier housing and/or the inside space 43 of the blister can preferably comprise a drying agent and/or protective gas. Hence, this has in particular a positive effect on the sterility and/or durability of the microarray.

FIG. 2b shows the carrier 10 of FIG. 2a in an applied state.

For application purposes, the blister 42 was detached or removed by a user, for example. Subsequently, the carrier 10 was connected to an application site 100 at the proximal end 17 of the carrier housing 14 via an adhesive layer 34, wherein the application site is in particular the skin of a user.

The carrier plunger 16 is in the application position. Here, the carrier plunger 16 has distally moved inside the carrier housing 14 and thus moved the microarray 12 out of the carrier housing 14 through the application opening 18. The microneedles 11 of the microarray 12 are inserted into the application site 100.

The carrier plunger 16 is held or fixed in a distal application position by a holding device 32. In the illustrated embodiment, the holding device 32 comprises a groove 33 which engages with the bead 31 of the carrier plunger 16. It is thus ensured that the carrier plunger 16 is further pressed and thus the microarray 12 is applied for an extended period of time.

The carrier plunger 16 was moved or deflected by access to or action upon the proximal end of the carrier plunger 16 via the access opening 28. This action was effected by the plunger 204 of an applicator 200 (see FIG. 7, for example), for example, which was in particular connected to the carrier 10 via the connecting device 20. Subsequently, a plunger 204 of the applicator 200 was accelerated, for example, and first struck the film 40 which was distally deflected due to its flexible configuration. In particular, the plunger 204 indirectly also struck the proximal end of the carrier plunger 16, whereby the releasing device 30 released the carrier plunger 16 for movement purposes. Then the carrier plunger 16 moved into the distal position illustrated in FIG. 2b and was fixed there by the holding device 32.

Instead of deflecting the carrier plunger 16 by means of the applicator 200, the former can be deflected or acted upon, preferably indirectly via the film 40, by means of the finger of the user pressing the proximal end of the carrier plunger 16.

The releasing device 30 is preferably configured as a target force triggering element. Thus, the carrier plunger is released only when at least a target force is applied, preferably when at least a target force is applied to the carrier plunger. Here, the target force triggering element can preferably be designed such that at least the required target force corresponds to an in particular optimum application force for puncturing by the and application of the microarray 12. In the illustrated configuration, the target force triggering element acts in particular via the detachable interlock between the groove 29 and the bead 31.

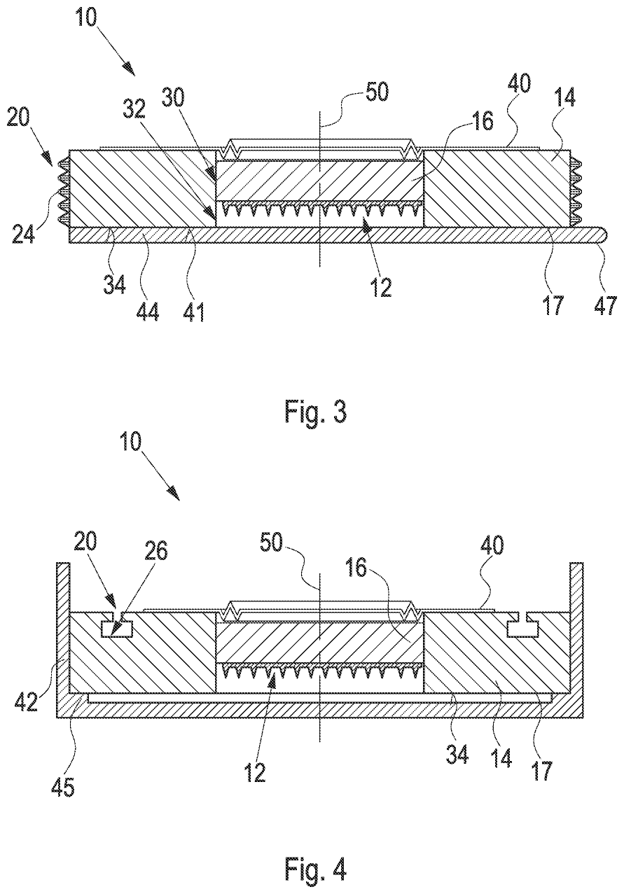

FIG. 3 shows another embodiment of a carrier 10. The illustrated embodiment is essentially similar to the embodiment of FIG. 2a.

In contrast to the embodiment of FIG. 2a, the releasing device 30 of the embodiment of FIG. 3 is designed as a fit, in particular an interference fit, between the carrier plunger 16 and the carrier housing 14. Due to the fit, the carrier plunger is thus first held in the initial position. After deflection of the carrier plunger 16, the fit assumes the function of the holding device 32 and thus ensures fixing of the carrier plunger in the application position.

The connecting device 20 comprises a thread 24 which can be connected with a corresponding mating thread of an applicator 200.

In contrast to the embodiment of FIG. 2a, the embodiment of FIG. 3 does not comprise a blister but a film 44. The film 44 is connected to the carrier housing 14 at the distal end 17, in particular via an adhesive layer 41. For example, the film 44 can be peeled off by a user using the illustrated film tab 47 and thus removed. The adhesive layer 41 also serves as a fixing means 34 such that the carrier 10 can thus be connected to an application site.

FIG. 4 shows another embodiment of a carrier 10. The embodiment is similar to the embodiment of FIG. 3, except for the differences described below.

The embodiment comprises a blister 42 which radially completely surrounds the carrier housing 14. At the proximal end 17 the carrier housing 14 abuts on a projection 45 radially extending from the blister 42 towards the inside. As seen from the point of view of the projection 45, the carrier housing 14 comprises at its distal end 17 an adhesive layer 34 which is located radially further inward and serves as a fixing means for fixing the carrier 10 to an application site. In contrast to other embodiments, for example the embodiment of FIG. 3, the adhesive layer 34 does not come into contact with the blister 42 but is spaced apart from the same.

The blister 42 constitutes a kind of guide at the proximal end, for example for an applicator 200. An applicator 200 can thus be pushed into the blister and then be connected to the connecting device 20 which is configured as a plug connection, as illustrated. Then it is possible that the blister 42, which is preferably merely slipped onto the carrier housing 14, is detached. Subsequently, an application based on the embodiments described above can be performed.

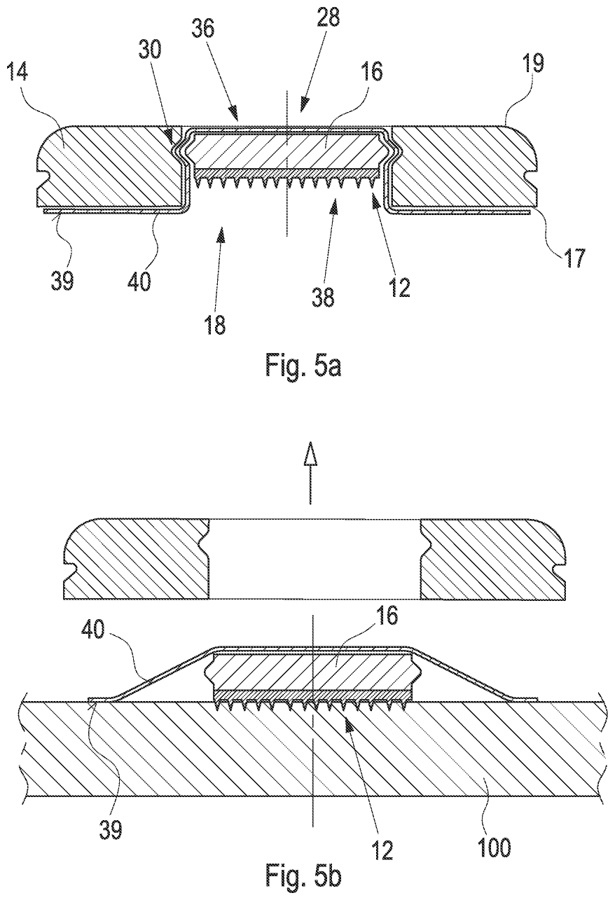

FIGS. 5a and 5b show another embodiment of a carrier 10. The embodiment does not comprise a holding device. After a movement of the carrier plunger 16, preferably with a resultant application of the microarray 12 to the application site 100, the carrier plunger 16 and the carrier housing 14 are thus no longer connected to each other and are separated from each other. Consequently, the carrier plunger 16 and the carrier housing 14 can be moved relative to each other.

FIG. 5b shows removal of the carrier housing 14 from the application site 100, wherein the carrier plunger 16 with an applied microarray remains on the application site 100.

The illustrated sterile barrier 36 comprises the film 40. In the initial position, as illustrated in FIG. 5a, the film 40 bulges from the distal end 17 of the carrier housing 14 through the application opening 18 and surrounds the carrier plunger 16 towards the access opening 28. Hence, a sterility of the inside 38 of the carrier housing or the microarray 12 is at least proximally ensured. Preferably, the film 40 distally comprises an adhesive layer 39 such that, as illustrated in FIG. 5b, the film 40 can be connected to the application site 100. After or during the application (FIG. 5b) the in particular elastic film 40 can either serve for further pressing the microarray 12 into the application site 100 and/or ensure sterility towards the environment.

In the embodiment of FIGS. 5a, 5b it is also possible to in particular removably provide a blister or another film at the distal end for ensuring sterility, preferably for closing an application opening 18.

The illustrated embodiments of the carrier 10 are configured such that they are rotationally symmetrical about a rotation axis 50. However, another configuration, for example as a cuboid, is feasible.

FIG. 6 shows another embodiment of a carrier 10. The illustrated embodiment essentially corresponds to the embodiment of FIG. 2a.

In contrast to the embodiment of FIG. 2a, the carrier plunger acting area 60 of the carrier plunger 16 of FIG. 6 is not plane but configured as a conical indentation, also referred to as a negative cone shape. For example, instead of the conical indentation a pyramidal or part-spherical indentation is feasible. It is also possible to configure other embodiments of the carrier 10, for example the embodiments of FIGS. 3 and 4, with a carrier plunger active area 60 as defined above.

The conical indentation of the carrier plunger acting area 60 of FIG. 6 advantageously causes an applicator plunger having a corresponding positive cone shape configuration, for example, to strike the carrier plunger acting area 60 such that an optimum force transmission is realized.

FIG. 7 shows an embodiment of an application system 300 according to the disclosure for applying a microarray with an embodiment of an applicator 200 according to the disclosure for applying a microarray as well as having a microarray device 10 which is configured here as a microarray disk. Here, the microarray carrier disk 10 is in particular configured with one, a plurality of or all features of the carrier configuration of FIG. 6.

The applicator 200 comprises an applicator housing 202 which, as illustrated, in particular includes a base body 203 (see FIG. 8a). The actuating device 212 and/or the holding device 208 can be considered part of the housing 202 or they can be identified as independent components.

The actuating device 212 comprises two actuators 214, 216.

The applicator 200 is reversibly connected to a microarray carrier disk 10 via the holding device 208 such that the microarray carrier disk 10 can optionally be received by the applicator 200 or detached from the same via the holding device operating member 230 configured as flexible lever arms.

FIG. 8a shows a sectional view of a first state of the application system 300 of FIG. 7, while FIG. 8b shows a second state.

The microarray carrier disk 10 is connected to the applicator 200 via a holding device 208 of the applicator 200. The holding device 208 comprises at least two flexible arms which can also referred to as snap-in hooks 230. The snap-in hooks 230 comprise projections 232 facing inward. These projections 232 are inserted into indentations 22 for connecting to the microarray carrier disk 10 and thus establish a connection. The connection between the holding device 208 and the microarray carrier disk 10 can also be referred to as tongue-and-groove joint, wherein the holding device 208 comprises at least one tongue 232 engaging with the groove 22 of the microarray carrier disk 10. The flexible arms 230 can be inwardly deflected or flexibly bent by a user, for example, whereby the tongue 232 exits the groove 22 and thus allows for an optional detachment of the microarray carrier device 10 from the applicator.

FIG. 8a shows an initial state of the applicator 200. The actuating device 212 comprises two separate mechanical actuators 214, 216. The mechanical actuator 214 is configured as a kind of pressing knob which can be displaced towards the base body 203 of the housing 202.

Furthermore, the embodiment of FIG. 8a comprises a pressing lever 216 as another actuator 216. The pressing lever 216 is rotatably supported and comprises a coupling element 242 at an inner end, which coupling element is coupled in a form-locking manner with a coupling element 240 of the plunger 204. The coupling elements 240, 242 jointly form part of a coupling mechanism 218 and in particular constitute the same. In the initial position the plunger 204 is held in a first position by this coupling mechanism. The plunger 204 comprises a conical plunger area for acting upon a carrier plunger acting area 60 of a microarray device 10.

FIG. 8b shows a second state of the applicator 200 of FIG. 8a, wherein the pressing knob 214 and the base body 203 relatively move towards each other. This movement towards each other releases a rotation of the pressing lever 216. Hence, the pressing lever 216 is rotated and thus the coupling element 242 of the pressing lever 216 is decoupled from the coupling element 240 of the plunger 204, whereby the plunger 204 is released for moving purposes.

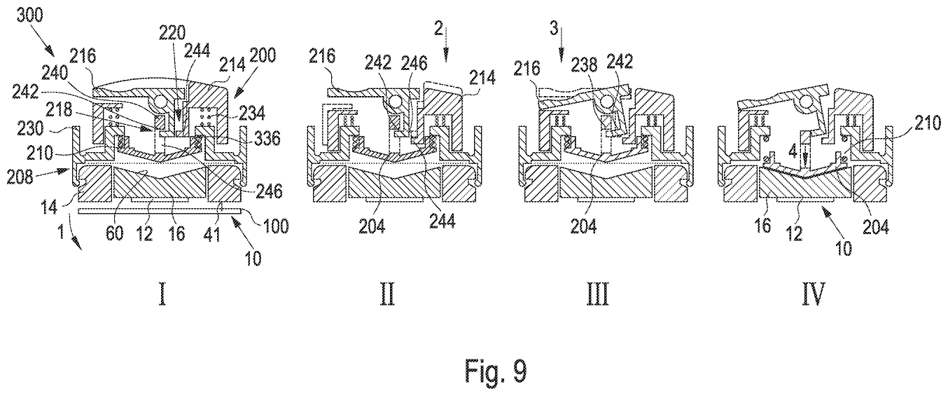

FIG. 9 shows various states of an application system 300 with an applicator 200, wherein the application system 300 and the applicator, respectively, essentially correspond to the configurations of FIGS. 7-8.

In the initial position I the application system 300 is placed onto an application site 100, such as the skin of a user, for example, as illustrated by a movement arrow 1. Here, it is preferred that the microarray carrier disk 10 is adhesively connected to the application site 100 via an adhesive layer 41. In the initial state I the microarray 12 of the microarray carrier device 10 is spaced apart from the skin 100.

In the initial state I the actuator 216 configured as a pressing lever 216 is blocked by a blocking element 244 of the other actuator 214 configured as a pressing knob 214. For this purpose, the blocking element 244 abuts on a blocking element 246 configured as a rear surface of the actuator 216 and thus prevents the actuator 216 from rotating.

The actuator 214 is held in a first position by a force defining device 234. In the illustrated embodiment, the force defining device 234 corresponds to a pretensioning element, in particular a spring 234. Here, the spring 234 is configured such that it allows for a movement of the actuator 214 in particular into the end position only when at least a predefined force is applied which in particular corresponds to the optimum pressing force exerted by the application system 300 onto the skin 100. The actuator 214 hence corresponds to a force triggering element which allows for actuation of the locking mechanism 220, i.e. unlocking, only when the predefined force or a larger (larger/equal) force as the predefined force is applied.

In state II the actuator 214 has been pressed as is illustrated by a movement arrow 2. Thus, the blocking element 244 of the actuator 214 is disengaged from the other actuator 216, in particular a blocking element 246 configured as a contact area 246. Thus, the locking mechanism 220 is actuated and, consequently, the applicator 200 is unlocked. The other actuator 216 configured as an operating element can hence be rotated.

The third state III shows such an operation of the operating element 216, wherein, as illustrated by a movement arrow 3, a pressure is exerted upon the pressing lever 216. Thereby, the coupling element 242 of the actuator 216 is disengaged from the coupling element 240 of the plunger 204 and hence an actuation of the coupling mechanism 238, i.e. decoupling, is performed. The plunger 204 is thus released for displacement purposes.

In the final state IV the plunger 204 is displaced, as illustrated by a movement arrow 4, via the pressing element 210 configured as a pretensioning device, in particular a spring. In particular, the conical plunger area 206 of the plunger 204 strikes the carrier plunger 12, in particular the carrier plunger acting area 60. Thereby, the carrier plunger 12 is displaced and hence the microarray 12 is applied.

Application by means of the illustrated applicator 200 is preferably performed only upon cumulative actuation of the actuating device 212, i.e. pressing the actuator 214 as well as operating the operating element 216.

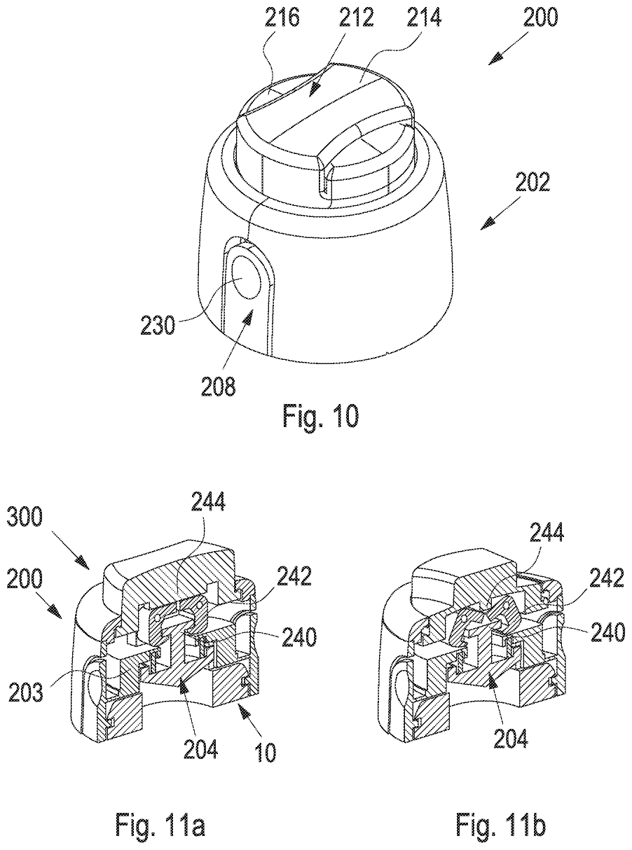

FIG. 10 shows another embodiment of an applicator 200 according to the disclosure.

The microarray carrier disk 10 is received by the holding device 208 in a manner similar to that of the embodiment of FIG. 7. In contrast to the embodiment of FIG. 7, the snap-in hooks 230 are configured such that they do not extend beyond the outer contour of the applicator 200.

The actuating device 212 comprises two actuators 214, 216 which, as illustrated, are integrally formed with each other, in particular as a rotary/pressing knob.

FIGS. 11a and 11b show two states of the embodiment of FIG. 10.

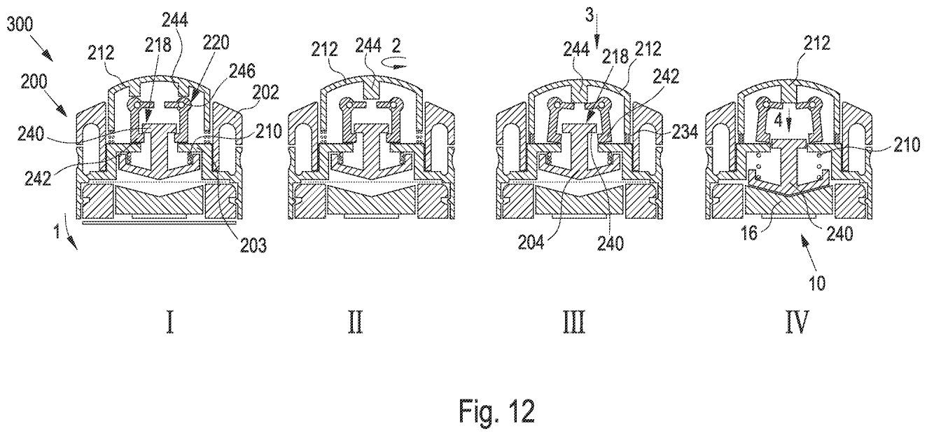

FIG. 12 shows an embodiment of an application system 300 according to the disclosure with an embodiment of an applicator 200 according to the disclosure, wherein the embodiments essentially correspond to the embodiments of FIG. 11a.

The applicator 200 comprises a locking mechanism 220 having at least one blocking element 244 (see FIG. 11a), preferably two blocking elements 244 (see FIG. 12). In the initial state the locking mechanism 220 prevents, via the at least one blocking element 244, the rotary/pressing knob from being pressed since the at least one blocking element 244 radially presses onto the axis of the retaining pawls 246 holding the plunger 204. Only after a rotation of preferably 90° of the rotary/pressing knob 212 (see movement arrow 2 in II) is the at least one blocking element 244 disengaged and hence the applicator 200 is unlocked.

By pressing the rotary/pressing knob 212 (see movement arrow 3 of III) against the force defining device 234 a rotation of the coupling elements 242, in particular configured as retaining pawls, is effected by the action of the blocking element 244 such that decoupling from the coupling elements 240 of the plunger 204 takes place. Thus, in III activation of the coupling mechanism 218, i.e. decoupling, takes place such that a displacement of the plunger 204 (see IV) is feasible.

In the embodiments of FIGS. 10-12, the operating element 216, by means of the rotating function of the rotary/pressing knob 212, actuates the locking mechanism 220. The force triggering element 214, by means of the pressing function of the rotary/pressing knob 212, activates the decoupling mechanism 218.

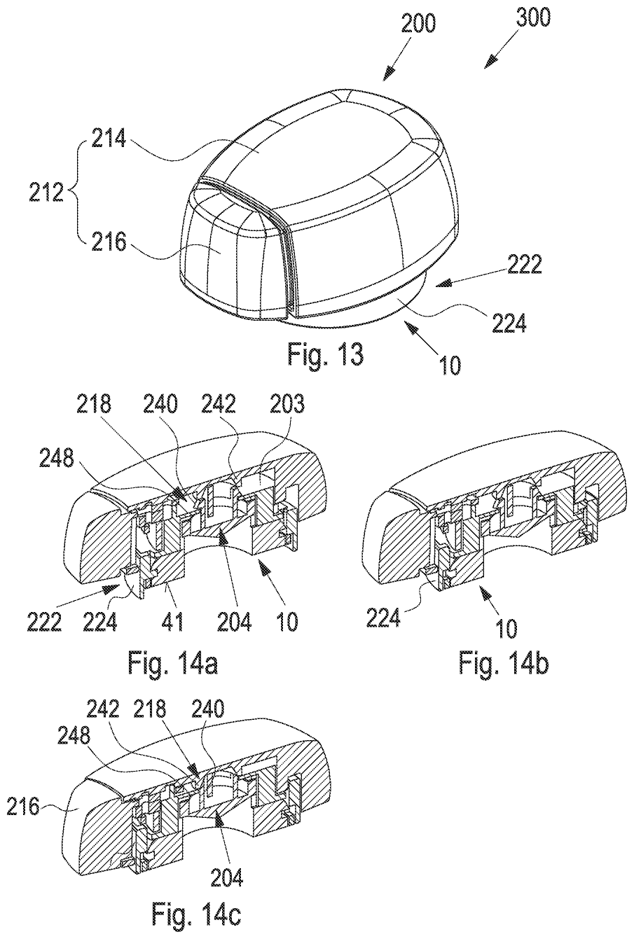

FIG. 13 shows another embodiment of an application system 300 according to the disclosure with an embodiment of an applicator 200 according to the disclosure, while the FIGS. 14a-14c show sectional views of various states of the embodiments of FIG. 13.

In contrast to the embodiments of FIGS. 7-12, the microarray carrier disk 10 is not reversibly but irreversibly received by the applicator 200 via a holding device 208 configured as a kind of bayonet closure.

A preferably spring-supported protecting device 222 is annularly arranged around the microarray carrier disk 10, which protecting device comprises a protecting collar 224 or is constituted by the same. In the initial state (see FIG. 14a) the protecting collar 224 is movably deflected in particular due to the pretension created by the pretensioning device (not shown) such that a contact of the adhesive layer 41 and/or a microarray 12 of the microarray carrier device 10 is shielded from the environment. Thereby, unintentional attaching of the microarray carrier device 10 to an object and/or contamination of the microarray 12 and/or physical action upon the microarray 12 etc. are advantageously prevented from occurring. Due to a vertical application of force to the applicator 200, for example to the skin of a user, the protecting collar 224, is displaced, preferably against a pretension, into the inside of the applicator 200. It is particularly preferred that the protecting collar is then in particular irreversibly held, for example snaps in and/or is hooked in. Thus, the adhesive layer 41 is cleared (see FIG. 14b).

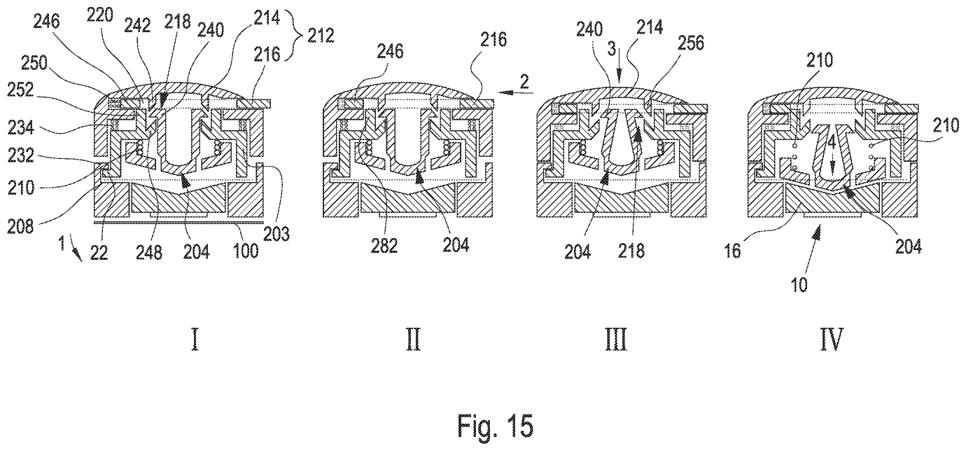

The locking mechanism 220 of the embodiment 13-15 comprises a gate valve 216 as an operating element. When the operating element 216 is displaced by a user, for example (see movement arrow 2 at II in FIG. 15), unlocking takes place, whereby a blocking element 252 of the base body 203 is released for movement purposes by displacing the locking element 246.

Subsequent pressing of the force triggering element 214 against the force defining device 234 (see movement arrow 3 of III in FIG. 15) leads to compression of the coupling element 240 of the plunger 204 via at least one beveled edge 256 of the force triggering element 214 and thus to decoupling of the coupling mechanism 218. Thus, the plunger 204 is released for movement by means of the pressing element 210 (see movement arrow 4 at IV in FIG. 15).

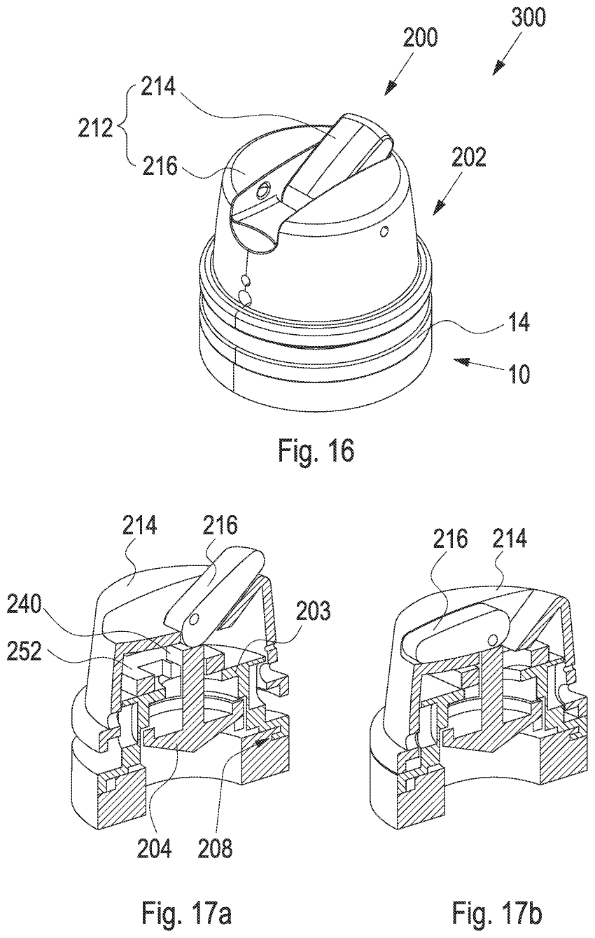

FIG. 16 shows another embodiment of an application system 300 according to the disclosure with another embodiment of an applicator 200 according to the disclosure.

FIGS. 17a and 17b show sectional views of various states of the embodiment of FIG. 16.

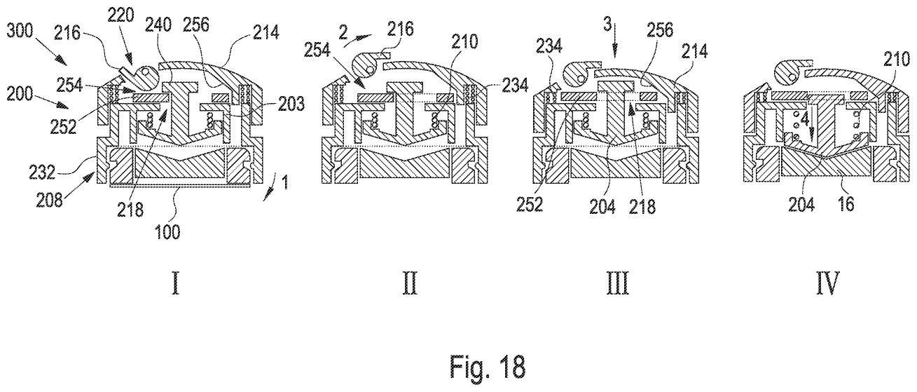

FIG. 18 shows another embodiment of an application system 300 with another embodiment of an applicator 200 according to the disclosure based on the embodiment of FIG. 16.

The microarray device 10 is received via a bayonet closure acting as a holding device 208. The locking mechanism 208 is activated by a toggle switch 216. In the first state (see FIG. 17a and I of FIG. 18, for example) the toggle switch 216 prevents, via an interlock with a blocking element 252, i.e. in particular a lack of distance 254, a movement of the pressing knob 214 corresponding to the force triggering element. By flipping the toggle switch 216 the interlock is released and a distance 254 is created (see II of FIG. 18). For example, when a user presses the force triggering device 214 against the force defining device 234, the blocking element 252 is displaced via the beveled edge 256 of the force triggering device 214 and thus release for displacing the plunger 204 is realized (see movement arrow 3 of III in FIG. 18). Thereby, the coupling mechanism 218 is activated, i.e. decoupled. Here, the blocking element 252 in particular corresponds to a template device through which the plunger 204 is fitted only in the case of a fitting arrangement, preferably by displacing the blocking element 252.

By means of the pressing element 210 the plunger 204 is then displaced for application purposes (see movement arrow 4 of IV in FIG. 18).

The various solution approaches, in particular the embodiments of FIGS. 7-18, can be combined with each other and/or exchanged for each other. In particular, the various holding devices (bayonet closure or snap-in connection, for example) can be used in all embodiments. Also, the various configurations of the actuators 214, 216 or the configurations of the force triggering elements and/or the operating elements can be variably used in all embodiments. In particular, the protecting device 222, particularly in the configuration of FIG. 14a, can be realized in all embodiments. Furthermore, preferably the coupling mechanism and/or the locking mechanism, in particular according to the various concepts of the embodiments of FIGS. 7-18, can be variably realized in all embodiments.

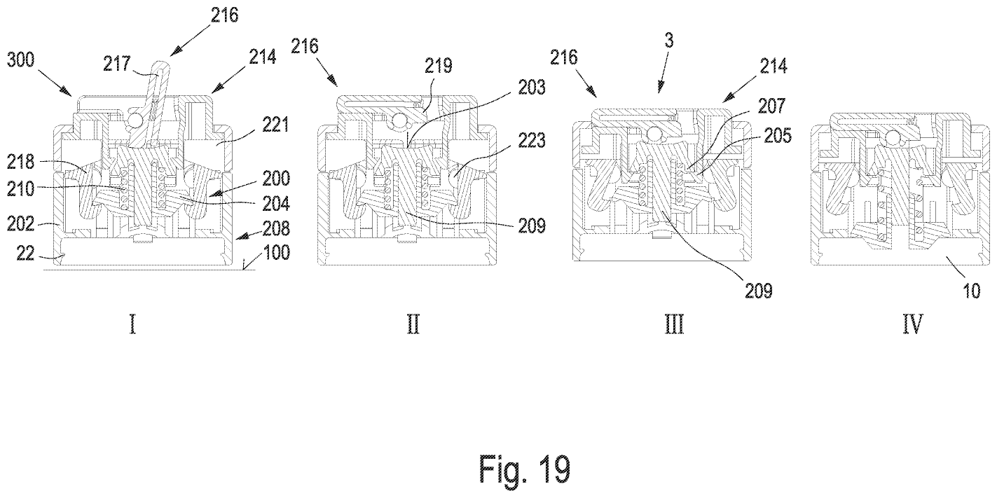

FIG. 19 shows another embodiment of an application system 300 with an applicator 200.

According to the embodiments described above, in the initial state I the application system 300 is placed onto an application site 100, i.e. in particular the skin. Similar to the other embodiments, the carrier 10 is held via a catching system comprising bosses 22. In the initial state I, the microarray held by the carrier 10 is spaced apart from the skin 100. In the initial state I the actuator 214, which is a large pressing knob in the illustrated exemplary embodiment, is blocked by a blocking element 216. The blocking element 216 is configured as a pivot lever 217. Since the pivot lever 217 abuts on the housing element 203, the actuator 214, in the initial position I, cannot be actuated, i.e. cannot be pressed down. Only when the pivot lever 217 is flipped, can the pressing knob be pressed down.

After release, the pivot lever 216 can be pivoted into state II. Since in state II a boss 219 of the actuator 216 no longer abuts on an upper side 203 of a guiding element 209, the two actuators 214, together with the pivot lever 216, can be pressed down, as illustrated by a movement arrow 3. On the one hand, this causes holding bosses 205 of the actuator 214 to engage behind holding bosses 207 of the guiding element 209. The holding bosses 205 fulfill the function of maintaining the triggered state and making another triggering impossible. It is thus obvious that triggering has taken place.

Further, due to actuation in the direction indicated by the movement arrow 3, the plunger 204 is released. This is realized by tilting the form-locking coupling mechanism 218 from position II into position III. For this purpose, the individual levers of the coupling mechanism 218 comprise spherical, in particular hemispherical bosses 223 serving as pivot bearings. By rotating the actuating element 218 and the cooperating inclined planes, the individual coupling elements 218 are tilted or pivoted.

Since the spring 210 is thus released, the plunger 204 is moved in the direction of the carrier element 10 carrying the microneedle array.

The Figures, in particular FIGS. 9, 12, 15, 18 and 19, first illustrate placement onto the application site (arrow 1) and subsequently actuation of the first actuator (arrow 2). Generally and in particular in the illustrated embodiments, it is preferred that first actuation of the first actuator and subsequently placement onto the application site take place, if possible. In other words, it is also possible to first perform unlocking and then placement.

1. An applicator for applying a microarray, comprising
a housing,
a plunger linearly movably arranged in the housing,
a holding device for receiving a microarray device,
a pressing element for displacing the plunger for acting upon a microarray device received by the holding device, and
an actuating device which holds the plunger in a first position and, by actuating the actuating device, releases the plunger for displacement purposes,
wherein the actuating device comprises at least two separate, in particular mechanical actuators, and
wherein release of the plunger by the actuating device takes place upon a cumulative actuation of the at least two actuators.

2. The applicator according to claim 1, wherein the actuation device comprises at least one force triggering element where actuation is performed when a defined force is applied.

3. The applicator according to claim 1, wherein the actuation device comprises at least one operating element where the actuation takes place when it is acted upon.

4. The applicator according to claim 2, wherein the force triggering element and/or the operating element comprise, in particular are constituted by:
a pressing and/or rotary knob, or
a lever, or
a slider, or
a switch.

5. The applicator according to claim 2, wherein the force triggering element and the operating element are in particular integrally formed with each other and are preferably configured as a rotary/pressing knob.

6. The applicator according to claim 1, wherein the actuating device comprises a form-locking and/or force-locking coupling mechanism which couples the plunger to the housing in a first state and decouples it from the housing in a second state for allowing the plunger to be displaced.

7. The applicator according to claim 6, wherein the actuating device comprises a form-locking and/or force-locking locking mechanism which blocks the coupling mechanism in a first deactivated state and unblocks it in a second activated state such that the coupling mechanism can allow the plunger to be displaced.

8. The applicator according to claim 6, wherein the force triggering element actuates the coupling mechanism or the locking mechanism.

9. The applicator according to claim 6, wherein the operating element actuates the coupling mechanism or the locking mechanism.

10. The applicator according to claim 1, wherein the holding device is reversibly detachably connectable or irreversibly connectable to the microarray device.

11. The applicator according to claim 1, further comprising a protecting device which is movable relative to the holding device from the environment, and in a second position releases it for application purposes.

12. The applicator according to claim 11, wherein the protecting device comprises a protecting collar wherein the protecting collar is held by a pretensioning device against a movement into the second position.

13. The applicator according to claim 1, further comprising a reversible or irreversible blocking device for preventing reuse of the applicator, wherein the blocking device fixes the plunger in a displaced position and/or blocks the actuating device.

14. An application system for applying a microarray, comprising
an applicator according to claim 1, and
a microarray device configured as a microarray carrier disk and comprising at least one microarray,
wherein the holding device receives the microarray device in a catching manner.

15. A method for displacing a plunger by means of an applicator according to claim 1,
wherein the plunger is held in a first position against a pretension,
wherein the plunger is released for displacement purposes by means of the pretension upon at least two separate actuations, and
wherein the at least two actuations for releasing purposes take place in a cumulative manner.
